(19) United States
(12) Patent Application Publication
(10) Pub. No.: US 2018/0185324 A1
(43) Pub. Date: Jul. 5, 2018

(54) NOVEL CANNABINOID COMBINATION THERAPIES FOR MULTIPLE MYELOMA (MM)

(71) Applicant: ONE WORLD CANNABIS LTD, Petach Tikva (IL)

(72) Inventors: Alon Sinai, Petach Tikva (IL); Ziv Turner, Petach Tikva (IL); Yehuda Baruch, Gedera (IL)

(73) Assignee: ONE WORLD CANNABIS LTD, Petach Tikva (IL)

(21) Appl. No.: 15/580,718

(22) PCT Filed: Jun. 9, 2016
(86) PCT No.: PCT/IL2016/050608

§ 371 Date: Dec. 8, 2017

Related U.S. Application Data
(60) Provisional application No. 62/173,992, filed on Jun. 11, 2015.

(51) Int. Cl. A61K 31/352; A61K 31/05; A61K 31/69; A61K 31/454; A61K 31/573; A61K 31/198; A61K 31/704; A61K 31/5377; A61K 36/185; A61K 45/06; A61P 35/00

(52) U.S. Cl. CPC A61K 31/352; A61K 31/05; A61K 31/69; A61K 31/454; A61K 31/573; A61K 2300/00; A61K 31/704; A61K 31/5377; A61K 36/185; A61K 45/06; A61P 35/00; A61K 31/198

(57) ABSTRACT

The present invention discloses a cytotoxic cocktail comprising; (a) a therapeutically effective amount of at least one cannabinoid selected from the group consisting of: cannabidiol (CBD) or a derivative thereof, Tetrahydrocannabinol (THC) or a derivative thereof, and any combination thereof; and (b) at least one therapeutic agent selected from the group consisting of: bortezomib (BTZ), carflizomib (CFZ), lenalidomide (LEN), dexamethasone (DEX), melphalan (MEL) and doxorubicin (DOXO). In a core embodiment the cocktail is conferring a synergistic effect with respect to inhibition or cytotoxicity of multiple myeloma (MM) cells, relative to said at least one therapeutic agent selected from the group consisting of: BTZ, CFZ, LEN, DEX, MEL, DOXO and said CBD and THC, administered separately in a similar concentration.

### FIELD OF THE INVENTION

The present invention relates to a method and composition for treating Multiple Myeloma (MM) comprising at least one cannabinoid. More specifically, the present invention pertains to a method and composition comprising the cannabinoids Tetrahydrocannabinol (THC) and/or cannabidiol (CBD).

### BACKGROUND OF THE INVENTION

Multiple myeloma (MM), also known as plasma cell myeloma, myelomatosis, or Kahler's, is a cancer of plasma cells, a type of white blood cell normally responsible for producing antibodies in which collections of abnormal plasma cells accumulate in the bone marrow, where they interfere with the production of normal blood cells. It is the second most common hematologic cancer as it accounts for 10% of all hematologic malignancies and represents 1% of all cancer diagnoses and 2% of all cancer deaths.

The disease develops in 6.1 per 100,000 people per year. It is more common in men and, for unknown reasons, is twice as common in African-Americans as it is in European-Americans. With conventional treatment, median survival is 3-4 years, which may be extended to 5-7 years or longer with advanced treatments. The five year survival rate is 45%. In multiple myeloma. Treatment results in significant survival benefits, however relapse is inevitable and the disease remains incurable.

Bone pain affects almost 70% of MM patients and is the most common symptom. Myeloma bone pain usually involves the spine and ribs, and worsens with activity. Persistent localized pain may indicate a pathological bone fracture. Involvement of the vertebrae may lead to spinal cord compression. Myeloma bone disease is due to the overexpression of Receptor Activator for Nuclear Factor κ B Ligand (RANKL) by bone marrow stroma. RANKL activates osteoclasts, which resorb bone. The resultant bone lesions are lytic in nature). The breakdown of bone also leads to release of calcium into the blood, leading to hypercalcemia and its associated symptoms.

MM is also commonly characterized in acute or chronic renal failure. The most common cause of renal failure is due to proteins secreted by the malignant cells. Myeloma cells produce monoclonal proteins of varying types, most commonly immunoglobulins and free light chains, resulting in abnormally high levels of these proteins in the blood. Depending on the size of these proteins, they may be excreted through the kidneys. Kidneys can be damaged by the tubulopathic effects of proteins or light chains. Increased bone resorption leads to hypercalcemia and causes nephrocalcinosis thereby also contributing to the renal failure. Amyloidosis is a distant third in the causation. Patients with Amyloidosis have high levels of Amyloid protein that can be excreted through the kidneys and cause damage to the kidneys and other organs. Other causes of renal failure in MM include hyperuricemia, recurrent infections and local infiltration of tumor cells.

Treatment for multiple myeloma is focused on therapies that decrease the clonal plasma cell population and consequently decrease the signs and symptoms of disease which includes alkylating agents, corticosteroids, proteasome inhibitors, and immunomodulatory drugs. In recent years, high-dose chemotherapy with autologous hematopoietic stem-cell transplantation has become the preferred treatment for patients under the age of 65.

Cannabinoids are a group of 21-carbon-containing terpenophenolic compounds produced uniquely by Cannabis species. They have been shown to have a protective effect against the development of certain types of tumors as well as to inhibit the growth and induce apoptosis of a broad spectrum of tumor cells. Antitumor effects are caused by various mechanisms, including induction of cell death, inhibition of cell growth, and inhibition of tumor angiogenesis invasion and metastasis.

So far, two cannabinoid-specific receptors, CB1 and CB2, have been characterized from mammalian tissues. They have been shown to possess anti-proliferative and anti-angiogenic effects in vitro as well as in vivo in different cancer models. Both cannabinoid systems are unambiguously osteo-protective, especially with regard to the aging skeleton. CB2 is expressed in osteoblasts and osteoclasts and stimulates bone formation. The endocannabinoid system also plays an important role in regulating skeletal remodeling and bone mass. These physiological processes are implicated in the development and progression of osteoporosis. Recently it has been discovered that the CB2 receptor is also highly expressed in MM cell lines.

Several patent applications recite compositions for treating myeloma which involves substrates of the cannabinoid-specific receptors. For example, patent application US patent app. No. 20130172388 recites Novel CB2 inverse agonists for treating multiple myeloma and osteoporosis bone diseases and Patent application WO2014057067 discloses the use of a combination of endocannabinois and cannabinoids complexes with a lipoprotein for the treatment of cancers dependent on hedgehog mechanisms of which MM is amongst them. The phsychotropic effect of these compositions is not yet known.

It is therefore a long felt and unmet need to formulate a composition comprising a CB2 agonist having no psychotropic effect as a novel therapeutic treatment for MM or for enhancing the efficacy of anticancer therapy used to treat the same.

### SUMMARY OF THE INVENTION

It is thus one object of the present invention to provide a cytotoxic cocktail comprising: (a) a therapeutically effective amount of at least one cannabinoid selected from the group consisting of: cannabidiol (CBD) or a derivative thereof, Tetrahydrocannabinol (THC) or a derivative thereof, and any combination thereof; and (b) at least one therapeutic agent selected from the group consisting of: bortezomib (BTZ), carfilzomib (CFZ), lenalidomide (LEN), dexamethasone (DEX), melphalan (MEL) and doxorubicin (DOXO); wherein said cocktail is conferring a synergistic effect with respect to inhibition or cytotoxicity of multiple myeloma (MM) cells, relative to said at least one therapeutic agent selected from the group consisting of: BTZ, CFZ, LEN, DEX, MEL, DOXO and said CBD and THC, administered separately in a similar concentration.

It is another object of the present invention to provide the cytotoxic cocktail as defined above, wherein said CBD and said THC are in a predefined ratio conferring inhibition or cytotoxicity of multiple myeloma (MM) cells relative to said CBD and said THC administered separately in a similar concentration.

It is another object of the present invention to provide the cytotoxic cocktail as defined in any of the above, wherein said CBD and said THC are in a predefined ratio conferring a synergistic effect with respect to inhibition or cytotoxicity of multiple myeloma (MM) cells relative to said CBD and said THC administered separately in a similar concentration.

It is another object of the present invention to provide the cytotoxic cocktail as defined in any of the above, wherein said CBD and said THC are administered in said predefined ratio of about 1:1.

It is another object of the present invention to provide the cytotoxic cocktail as defined in any of the above, wherein said CBD and said THC are administered in said predefined ratio of about 1:5, respectively.

It is another object of the present invention to provide the cytotoxic cocktail as defined in any of the above, wherein said CBD and said THC are administered in said predefined ratio of about 5:1, respectively.

It is another object of the present invention to provide the cytotoxic cocktail as defined in any of the above, wherein said CBD and said THC are administered in said predefined ratio of about 5:2, respectively.

It is another object of the present invention to provide the cytotoxic cocktail as defined in any of the above, wherein said cytotoxic or inhibition effect is defined as significantly decreased viability of RPMIS multiple myeloma (MM) cells in vitro.

It is another object of the present invention to provide the cytotoxic cocktail as defined in any of the above, wherein said CBD and said THC have a combination index (CI) value lower than 1 indicating synergism.

It is another object of the present invention to provide the cytotoxic cocktail as defined in any of the above, wherein said at least one of BTZ, CFZ, LEN, DEX, MEL and DOXO in said cocktail, decreases the viability of MM cells, in a dose dependent manner.

It is another object of the present invention to provide the cytotoxic cocktail as defined in any of the above, wherein said decrease in viability is defined as decreased viability of at least 50% of RPMIS multiple myeloma (MM) cells in vitro.

It is another object of the present invention to provide the cytotoxic cocktail as defined in any of the above, wherein said synergistic effect is defined as at least 50% inhibition of RPMIS multiple myeloma (MM) cells in vitro.

It is another object of the present invention to provide the cytotoxic cocktail as defined in any of the above, wherein the concentration of said CBD is in the range of about 2% to about 20%.

It is another object of the present invention to provide the cytotoxic cocktail as defined in any of the above, wherein the concentration of said THC or said derivative thereof is in the range of about 2% to about 20%.

It is another object of the present invention to provide the cytotoxic cocktail as defined in any of the above, wherein said composition is adapted to be administered in a route selected from a group consisting of: intranasal, transdermal, intravenous, oral, and any combination thereof.

It is another object of the present invention to provide the cytotoxic cocktail as defined in any of the above, wherein said composition is adapted for oral administration in a formulation selected from a group of preparations consisting of syrup, drops, tincture, tablet, capsule, solution, emulsion, suspension, granules, powder, and any combination thereof.

It is another object of the present invention to provide the cytotoxic cocktail as defined in any of the above, wherein said composition is adapted to be administered in combination with an additional MM therapeutic agent.

It is another object of the present invention to provide the cytotoxic cocktail as defined in any of the above, wherein said additional MM therapeutic agent is selected from a group consisting of alkylating agents, corticosteroids, proteasome inhibitors, immunomodulatory drugs, and any combination thereof.

It is another object of the present invention to provide the cytotoxic cocktail as defined in any of the above, wherein said CBD or said derivative thereof interacts with at least one receptor selected from a group consisting of Cannabinoid receptor type 1 (CB1), Cannabinoid receptor type 2 (CB2), and any combination thereof.

It is another object of the present invention to provide the cytotoxic cocktail as defined in any of the above, wherein said THC or said derivative thereof interacts with at least one receptor selected from a group consisting of Cannabinoid receptor type 1 (CB1), Cannabinoid receptor type 2 (CB2), and any combination thereof.

It is another object of the present invention to provide the cytotoxic cocktail as defined in any of the above, wherein said composition additionally comprises inactive ingredients selected from a group consisting of antiadherants, binders, coatings, disintegrants, flavours, colours, lubricants, glidants, sorbents, preservatives, sweeteners, and any combination thereof.

It is another object of the present invention to provide the cytotoxic cocktail as defined in any of the above, wherein said composition is in a sustained release dosage form; said sustained release dosage form is selected from a group consisting of liposomes, drug polymer conjugates, microencapsulation, controlled-release tablet coating, and any combination thereof.

It is another object of the present invention to provide the cytotoxic cocktail as defined in any of the above, wherein said composition is nonpsychoactive.

It is another object of the present invention to provide the cytotoxic cocktail as defined in any of the above, wherein said composition is administered once, twice, three or four times through the day.

It is another object of the present invention to provide the cytotoxic cocktail as defined in any of the above, wherein said CBD and/or said THC is obtained from at least one cannabis plant.

It is another object of the present invention to provide the cytotoxic cocktail as defined in any of the above, wherein said cannabis plant is a CBD rich strain.

It is another object of the present invention to provide the cytotoxic cocktail as defined in any of the above, wherein said CBD rich strain is selected from a group consisting of Avidekel, Fedora 17, ACDC, and any combination thereof.

It is another object of the present invention to provide the cytotoxic cocktail as defined in any of the above, wherein said cannabis plant is a THC rich strain.

It is another object of the present invention to provide the cytotoxic cocktail as defined in any of the above, wherein said THC rich strain is selected from a group consisting of Black Destroyer, Critical Neville Haze, Mataro Blue, LSD OG Kush, Pineapple Chunk, Blue Monster Holk, Y Griega, Satori, Tutankhamon, and any combination thereof.

It is another object of the present invention to provide the cytotoxic cocktail as defined in any of the above, wherein said composition comprises a cannabis extract or cannabis oil.

It is another object of the present invention to provide the cytotoxic cocktail as defined in any of the above, wherein said THC or derivative thereof and/or said CBD or derivative thereof is derived from a cannabis extract.

It is another object of the present invention to provide the cytotoxic cocktail as defined in any of the above, wherein said CBD or derivative thereof is produced by a synthetic route.

It is another object of the present invention to provide the cytotoxic cocktail as defined in any of the above, wherein said THC or derivative thereof is produced by a synthetic route.

It is another object of the present invention to provide the cytotoxic cocktail as defined in any of the above, wherein said composition is dissolved in a lipophilic solvent or suspension carrier.

It is another object of the present invention to provide the cytotoxic cocktail as defined in any of the above, wherein said lipophilic solvent or suspension carrier are selected from a group consisting of medium-chain triglyceride, short-chain triglyceride, medium-chain partial glyceride, polyoxyethylated fatty alcohol, polyoxyethylated fatty acid, polyoxyethylated fatty acid triglyceride or partial glyceride, ester of fatty acids with low molecular weight alcohols, a partial ester of sorbitan with fatty acids, a polyoxyethylated partial ester of sorbitan with fatty acids, a partial ester of sugars or oligomeric sugars with fatty acids, a polyethylene glycol, lecithin, vegetable oil, and any combination thereof.

It is another object of the present invention to provide the cytotoxic cocktail as defined in any of the above, wherein said cocktail decreases significantly the viability of RPMI8226 cells in comparison to BTZ, CFZ, CBD and THC alone.

It is another object of the present invention to provide the cytotoxic cocktail as defined in any of the above, wherein the combination of CBD: THC (1:1) with BTZ, CFZ, DOXO and MEL results in a significantly increased cytotoxicity effect as compared to BTZ, CFZ, DOXO, MEL, CBD and THC alone.

It is another object of the present invention to provide the cytotoxic cocktail as defined in any of the above, wherein the combination of CBD or CBD: THC (1:1; 5:1, 5:2 respectively) with BTZ or CFZ decreases significantly the viability of RPMI8226 cells in comparison with BTZ, CFZ, CBD and THC alone.

It is another object of the present invention to provide acytotoxic cocktail comprising: (a) an effective amount of at least one cannabinoid selected from the group consisting of: cannabidiol (CBD) or a derivative thereof, Tetrahydrocannabinol (THC) or a derivative thereof and any combination thereof; and (b) at least one therapeutic agent selected from the group consisting of bortezomib (BTZ), carfilzomib (CFZ), lenalidomide (LEN), dexamethasone (DEX), melphalan (MEL) and doxorubicin (DOXO); said CBD and said THC are in a predefined ratio conferring a synergistic effect with respect to inhibition of multiple myeloma (MM) cells relative to said CBD and said THC administered separately in a similar concentration; wherein said cocktail confers a synergistic effect with respect to inhibition or cytotoxicity of multiple myeloma (MM) cells relative to said at least one of BTZ, CFZ, LEN, DEX, MEL, DOXO and said CBD and THC, administered separately in a similar concentration.

It is another object of the present invention to provide the cytotoxic cocktail as defined in any of the above, wherein said CBD and said THC are administered in a ratio selected from the group consisting of: about 1:1, 5:1, 1:5 and 5:2 respectively.

It is another object of the present invention to provide the cytotoxic cocktail as defined in any of the above, wherein said cocktail decreases significantly the viability of RPMI8226 cells in comparison with BTZ, CFZ, CBD and THC alone.

It is another object of the present invention to provide the cytotoxic cocktail as defined in any of the above, wherein the combination of CBD: THC (1:1) with BTZ, CFZ, DOXO and MEL results in a significantly increased cytotoxicity effect as compared to BTZ, CFZ, DOXO, MEL, CBD and THC alone.

It is another object of the present invention to provide the cytotoxic cocktail as defined in any of the above, wherein the combination of CBD or CBD: THC (1:1; 5:1, 5:2 respectively) in combination with BTZ or CFZ decreased significantly the viability of RPMI8226 cells in comparison with BTZ, CFZ, CBD and THC alone.

It is another object of the present invention to provide a method of treating multiple myeloma (MM) in a subject; said method comprising administrating to the subject a therapeutically effective amount of a cytotoxic cocktail comprising: (a) a therapeutically effective amount of at least one cannabinoid selected from a group consisting of: cannabidiol (CBD) or a derivative thereof, Tetrahydrocannabinol (THC) or a derivative thereof, and any combination thereof, and (b) at least one therapeutic agent consisting of bortezomib (BTZ), carfilzomib (CFZ), lenalidomide (LEN), dexamethasone (DEX), melphalan (MEL) and doxorubicin (DOXO); wherein said cocktail is conferring a synergistic effect with respect to inhibition or cytotoxicity of multiple myeloma (MM) cells relative to said at least one of BTZ, CFZ, LEN, DEX, MEL, DOXO, CBD and THC administered separately in a similar concentration.

It is another object of the present invention to provide the method as defined in any of the above, additionally comprising step of providing said CBD and said THC in a predefined ratio of about 1:5 or 5:1, 5:2 or 1:1, respectively.

It is another object of the present invention to provide the method as defined in any of the above, wherein said step of administrating said CBD and said THC in said predefined ratio confers a synergistic effect with respect to inhibition of multiple myeloma (MM) cells relative to said CBD and said THC when administered separately in a similar concentration.

It is another object of the present invention to provide the method as defined in any of the above, wherein said step of administrating said at least one of BTZ, CFZ, LEN, DEX, MEL and DOXO significantly decreases the viability of MM cells in a dose dependent manner.

It is another object of the present invention to provide the method as defined in any of the above, additionally comprising steps of providing said extract with CBD concentration in the range of about 2% to about 20%.

It is another object of the present invention to provide the method as defined in any of the above, additionally comprising steps of providing said extract with THC concentration in the range of about 2% to about 20%.

It is another object of the present invention to provide the method as defined in any of the above, additionally comprising steps of administering said composition in a route selected from a group consisting of: intranasal, transdermal, intravenous, oral, and any combination thereof.

It is another object of the present invention to provide the method as defined in any of the above, additionally comprising steps of administering said composition orally in a formulation selected from a group of preparations consisting of syrup, drops, tincture, tablet, capsule, solution, emulsion, suspension, granules, powder, and any combination thereof.

It is another object of the present invention to provide the method as defined in any of the above, additionally comprising steps of administering said composition in a manner selected from a group consisting of: intranasal, transdermal, intravenous, oral, and any combination thereof.

It is another object of the present invention to provide the method as defined in any of the above, additionally comprising steps of administering said composition over a time period of about 1 day to about 6 months.

It is another object of the present invention to provide the method as defined in any of the above, additionally comprising steps of administering said composition in a dosage of CBD of up to 400 mg per day, preferably in the range of about 2 mg to about 400 mg per day.

It is another object of the present invention to provide the method as defined in any of the above, additionally comprising steps of administering said composition in a dosage of THC of up to 400 mg per day, preferably in the range of about 10 mg to about 400 mg per day.

It is another object of the present invention to provide the method as defined in any of the above, additionally comprising steps of administering said composition once, twice, three or four times through the day.

It is another object of the present invention to provide the method as defined in any of the above, additionally comprising steps of administering said composition with an additional MM therapeutic agent.

It is another object of the present invention to provide the method as defined in any of the above, selecting said additional MM therapeutic agent is selected from a group consisting of alkylating agents, corticosteroids, proteasome inhibitors, immunomodulatory drugs, and any combination thereof.

It is another object of the present invention to provide the method as defined in any of the above, additionally comprising steps of formulating said composition with an inactive ingredient selected from a group consisting of antiadherents, binders, coatings, disintegrants, flavours, colours, lubricants, glidants, sorbents, preservatives, sweeteners, and any combination thereof.

It is another object of the present invention to provide the method as defined in any of the above, additionally comprising steps of formulating said composition in a sustained release dosage form; said sustained release dosage form is selected from a group consisting of liposomes, drug polymer conjugates, microencapsulation, controlled-release tablet coating, and any combination thereof.

It is another object of the present invention to provide the method as defined in any of the above, wherein said administration does not significantly cause a psychoactive effect.

It is another object of the present invention to provide the method as defined in any of the above, additionally comprising steps of administering said CBD with Tetrahydrocannabinol (THC) in a concentration which is equal or less than 2%.

It is another object of the present invention to provide the method as defined in any of the above, wherein said step of administering said cocktail decreases significantly the viability of RPMI8226 cells in comparison with BTZ, CFZ, CBD and THC alone.

It is another object of the present invention to provide the method as defined in any of the above, wherein the combination in said cocktail is of CBD: THC (1:1) with BTZ, CFZ, DOXO and MEL results in a significantly increased cytotoxicity as compared to BTZ, CFZ, DOXO, MEL, CBD and THC alone.

It is another object of the present invention to provide the method as defined in any of the above, wherein the combination in said cocktail is of CBD or CBD: THC (1:1; 5:1, 5:2 respectively) with BTZ or CFZ decreased significantly the viability of RPMI8226 cells in comparison with BTZ, CFZ, CBD and THC alone.

It is another object of the present invention to provide a method of treating multiple myeloma (MM) in a subject; said method comprising administrating to said subject a therapeutically effective amount of cytotoxic cocktail characterized by: (a) at least one cannabinoid selected from the group consisting of: cannabidiol (CBD) or a derivative thereof, Tetrahydrocannabinol (THC) or a derivative thereof, and any combination thereof; and (b) at least one therapeutic agent selected from the group consisting of bortezomib (BTZ), carfilzomib (CFZ), lenalidomide (LEN), dexamethasone (DEX), melphalan (MEL) and doxorubicin (DOXO); said THC and said CBD are administered in a predefined ratio providing a synergetic effect with respect to inhibition of multiple myeloma (MM) cells relative to said CBD and said THC when administered separately in a similar concentration; wherein said cocktail conferring a synergistic effect with respect to inhibition or cytotoxicity of multiple myeloma (MM) cells relative to said at least one of BTZ, CFZ, LEN, DEX, MEL, DOXO, administered separately in a similar concentration.

It is another object of the present invention to provide the method as defined in any of the above, wherein said predefined ratio is of about 1:5 or 5:1 or 5:2 or 1:1, respectively.

It is another object of the present invention to provide the method as defined in any of the above, wherein said step of administering said cocktail decreases significantly the viability of RPMI8226 cells in comparison with BTZ, CFZ, CBD and THC alone.

It is another object of the present invention to provide the method as defined in any of the above, wherein the combination in said cocktail is of CBD: THC (1:1) with BTZ, CFZ, DOXO and MEL results in a significantly increased cytotoxicity as compared to BTZ, CFZ, DOXO, MEL, CBD and THC alone.

It is another object of the present invention to provide the method as defined in any of the above, wherein the combination in said cocktail is of CBD or CBD: THC (1:1; 5:1, 5:2 respectively) in combination with BTZ or CFZ decreased significantly the viability of RPMI8226 cells in comparison with BTZ, CFZ, CBD and THC alone.

It is another object of the present invention to provide a method for inhibiting multiple myeloma (MM) cells by administering a therapeutic dose of a cytotoxic cocktail as defined in any of the above, to human MM cells.

It is another object of the present invention to provide the method as defined in any of the above, wherein said MM cells are in vitro.

It is another object of the present invention to provide the method as defined in any of the above, wherein said MM cells are ex vivo.

It is another object of the present invention to provide the method as defined in any of the above, wherein said MM cells are in situ.

It is another object of the present invention to provide a use of a cytotoxic cocktail comprising: (a) a therapeutically effective amount of, or an extract consisting of essentially a therapeutically effective amount of at least one cannabinoid selected from the group consisting of: cannabidiol (CBD) or a derivative thereof, Tetrahydrocannabinol (THC) or a derivative thereof, and any combination thereof; and (b) at least one therapeutic agent consisting of bortezomib (BTZ), carfilzomib (CFZ), lenalidomide (LEN), dexamethasone (DEX), melphalan (MEL) and doxorubicin (DOXO); in the manufacture of a medicament to treat multiple myeloma (MM); wherein said cocktail is conferring a synergistic effect with respect to inhibition or cytotoxicity of multiple myeloma (MM) cells, relative to said at least one therapeutic agent selected from the group consisting of: BTZ, CFZ, LEN, DEX, MEL, DOXO and said CBD and THC, administered separately in a similar concentration.

It is another object of the present invention to provide the use as defined in any of the above, additionally comprising steps of providing said extract with CBD concentration in the range of about 2% to about 20%.

It is another object of the present invention to provide the use as defined in any of the above, additionally comprising steps of providing said extract with THC concentration in the range of about 2% to about 20%.

It is another object of the present invention to provide the use as defined in any of the above, additionally comprising steps of administering said composition in a route selected from a group consisting of: intranasal, transdermal, intravenous, oral, and any combination thereof.

It is another object of the present invention to provide the use as defined in any of the above, additionally comprising steps of administering said composition orally in a formulation selected from a group of preparations consisting of syrup, drops, tincture, tablet, capsule, solution, emulsion, suspension, granules, powder, and any combination thereof.

It is another object of the present invention to provide the use as defined in any of the above, additionally comprising steps of administering said composition in a manner selected from a group consisting of: intranasal, transdermal, intravenous, oral, and any combination thereof.

It is another object of the present invention to provide the use as defined in any of the above, additionally comprising steps of administering said composition over a time period of about 1 day to about 6 months.

It is another object of the present invention to provide the use as defined in any of the above, additionally comprising steps of administering said composition in a dosage of CBD of up to 400 mg per day, preferably in the range of about 100 mg to about 400 mg per day.

It is another object of the present invention to provide the use as defined in any of the above, additionally comprising steps of administering said composition in a dosage of THC of up to 400 mg per day, preferably in the range of about 100 mg to about 400 mg per day.

It is another object of the present invention to provide the use as defined in any of the above, additionally comprising steps of administering said composition once, twice, three or four times through the day.

It is another object of the present invention to provide the use as defined in any of the above, additionally comprising steps of administering said composition with an additional MM therapeutic agent.

It is another object of the present invention to provide the use as defined in any of the above, further comprising steps of selecting said additional MM therapeutic agent from a group consisting of alkylating agents, corticosteroids, proteasome inhibitors, immunomodulatory drugs, and any combination thereof.

It is another object of the present invention to provide the use as defined in any of the above, additionally comprising steps of formulating said composition with an inactive ingredient selected from a group consisting of antiadherents, binders, coatings, disintegrants, flavours, colours, lubricants, glidants, sorbents, preservatives, sweeteners, and any combination thereof.

It is another object of the present invention to provide the use as defined in any of the above, additionally comprising steps of formulating said composition in a sustained release dosage form; said sustained release dosage form is selected from a group consisting of liposomes, drug polymer conjugates, microencapsulation, controlled-release tablet coating, and any combination thereof.

It is another object of the present invention to provide the use as defined in any of the above, wherein said administration does not cause a psychoactive effect.

It is another object of the present invention to provide the use as defined in any of the above, additionally comprising steps of administering said CBD with Tetrahydrocannabinol (THC) in a concentration which is equal or less than 2%.

It is another object of the present invention to provide the use as defined in any of the above, wherein said CBD and said THC administered in a predefined ratio conferring a synergistic effect with respect to inhibition of multiple myeloma (MM) cells relative to said CBD and said THC administered separately in a similar concentration.

It is another object of the present invention to provide the use as defined in any of the above, wherein said CBD and said THC are administered in a ratio of about 1:5 or 5:1 or 5:2 or 1:1, respectively

It is another object of the present invention to provide the use as defined in any of the above, wherein said synergistic effect is defined as at least 50% inhibition on RPMI8226 multiple myeloma (MM) cells in vitro.

It is another object of the present invention to provide the use as defined in any of the above, wherein said synergistic effect is defined as more than about 80% inhibition on RPMI8226 multiple myeloma (MM) cells in vitro.

It is another object of the present invention to provide the use as defined in any of the above, wherein said CBD and said THC have a combination index (CI) value of less than 1 indicating synergism.

It is another object of the present invention to provide the use as defined in any of the above, wherein said cocktail also decreases significantly the viability of RPMI8226 cells in comparison with BTZ, CFZ, CBD and THC alone.

It is another object of the present invention to provide the use as defined in any of the above, wherein said at least one of BTZ, CFZ, LEN, DEX, MEL and DOXO significantly decreases the viability of MM cells as compared to said BTZ, CFZ, LEN, DEX, MEL and DOXO administered separately in a similar concentration, in a dose dependent manner.

It is another object of the present invention to provide the use as defined in any of the above, wherein said decrease in viability is defined as decreased viability of at least 50% of RPMIS multiple myeloma (MM) cells in vitro.

It is another object of the present invention to provide the use as defined in any of the above, wherein said cocktail also decreases significantly the viability of RPMI8226 cells in comparison with BTZ, CFZ, CBD and THC alone.

It is another object of the present invention to provide the use as defined in any of the above, wherein the combination of CBD: THC (1:1) with BTZ, CFZ, DOXO and MEL results in a significantly increase cytotoxicity as compared to BTZ, CFZ, DOXO, MEL, CBD and THC alone.

It is another object of the present invention to provide the use as defined in any of the above, wherein the combination of CBD or CBD: THC (1:1; 5:1, 5:2 respectively) in combination with BTZ or CFZ decreased significantly the viability of RPMI8226 cells in comparison with BTZ, CFZ, CBD and THC alone.

### BRIEF DESCRIPTION OF THE FIGURES

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

FIG. 1 is a diagram representing the results of an experiment examining the effect of different compositions on the viability of MM cells, as an embodiment of the present invention;

FIG. 2 is a graph representing RPMIS MM cell line survival (%) vs. concentration (μM) of CBD, THC and their combinations, as an embodiment of the present invention;

FIG. 3 is a graph representing the combinatorial effect of CBD with THC;

FIG. 4 is a graph representing the combinatorial cytotoxic effect of CBD, THC, CBD: THC (1:1; 5:1, 1:5 and 5:2 respectively) in combination with BTZ on RPMI8266 MM cells;

FIG. 5 is a graph representing the combinatorial cytotoxic effect of CBD, THC, CBD: THC (1:1; 5:1 and 1:5 respectively) in combination with CFZ on RPMI8266 MM cells;

FIG. 6 is a graph representing the combinatorial cytotoxic effect of CBD, THC, CBD: THC (1:1; 5:1 and 1:5 respectively) in combination with DEX on RPMI8266 MM cells;

FIG. 7 is a graph representing the combinatorial cytotoxic effect of CBD, THC, CBD: THC (1:1; 5:1, 1:5 and 5:2 respectively) in combination with DOXO on RPMI8266 MM cells; and

FIG. 8 is a graph representing the combinatorial cytotoxic effect of CBD, THC, CBD: THC (1:1; 5:1, 1:5 and 5:2 respectively) in combination with MEL on RPMI8266 MM cells.

### DETAILED DESCRIPTION OF THE PREFERRED EMBODIMENTS

The essence of the present invention is to provide a composition for treating multiple myeloma (MM) comprising cannabidiol (CBD) and/or Tetrahydrocannabinol (THC) or any extract thereof. More specifically, the present invention recites a composition comprising cannabis extracts.

As used herein the term “about” denotes ±25% of the defined amount or measure or value.

The term “multiple myeloma (MM)” refers hereinafter to a cancer of plasma cells. In multiple myeloma, collections of abnormal plasma cells accumulate in the bone marrow, where they interfere with the production of normal blood cells. Most cases of multiple myeloma also feature the production of a paraproteinan—an abnormal antibody which can cause kidney problems. Bone lesions and hypercalcemia (high blood calcium levels) are also often encountered. MM is also known as plasma cell myeloma, myelomatosis, or Kahler's disease.

The term “multiple myeloma cells” or “MM cells” as used herein refers to cell lines (of abnormal plasma cells) derived from MM subjects.

The term “inhibition of multiple myeloma cells” or “inhibition of MM cells” as used herein refers to an anti-MM effect including decrease in survival rate of MM cells, cytotoxic effect on MM cells, tumor size reduction, reduced viability of MM cells, apoptosis, cell cycle arrest, cell signaling arrest, mitochondrial trans membrane potential arrest and ROS production arrest.

The term “cannabidiol (CBD)” refers hereinafter to one of at least 85 active cannabinoids identified in cannabis. Cannabidiol is a major phytocannabinoid, accounting for up to 40% of the plant's extract. CBD is considered to have a wider scope of medical applications than Tetrahydrocannabinol (THC). Cannabidiol has a very low affinity for CB1 and CB2 receptors but acts as an indirect antagonist of their agonists. CBD may potentiate THC's effects by increasing CB1 receptor density or through another CB1-related mechanism. It is also an inverse agonist of CB2 receptors. CBD possesses antiproliferative, pro-apoptotic effects and inhibits cancer cell migration, adhesion and invasion.

The term “Tetrahydrocannabinol (THC)” refers hereinafter to the principal psychoactive constituent (or cannabinoid) of the cannabis plant. THC has a partial agonist activity at the cannabinoid receptor CB1 and the CB2 receptor.

The term “THC rich cannabis strain” refers hereinafter to a cannabis strain having 20% or more THC. More specifically the term relates but is not limited to the following strains: Black Destroyer, Critical Neville Haze, Mataro Blue, LSD OG Kush, Pineapple Chunk, Blue Monster Holk, Y Griega, Satori, Tutankhamon.

The term “CBD rich cannabis strain” refers hereinafter to a cannabis strain having 1% or more CBD. More specifically the term relates but is not limited to the following strains: Avidekel, Fedora 17, ACDC.

The term “Avidekel” refers hereinafter to a cannabis strain comprising 15.8% CBD and less than 1% THC which may be found in patent application US 2014/0259228.

The term “Fedora 17” refers hereinafter to a cannabis strain having a cannabionoid profile consistently around 1% CBD with THC less than 0.1%.

The term “ACDC” refers hereinafter to a cannabis strain having about 19% CBD and a THC/CBD ration of about 1:20.

The term “cannabinoid receptor” refers hereinafter to a class of cell membrane receptors under the G protein-coupled receptor superfamily. There are currently two known subtypes of cannabinoid receptors, termed CB1 and CB2. The CB1 receptor is expressed mainly in the brain, but also in the lungs, liver and kidneys. The CB2 receptor is expressed mainly in the immune system and in hematopoietic cells.

The term “Cannabinoid receptor type 1 (CB1)” refers hereinafter to a G protein-coupled cannabinoid receptor located primarily in the central and peripheral nervous system. It is activated by the endocannabinoid neurotransmitters anandamide and 2-arachidonoyl glyceride (2-AG); by plant cannabinoids, such as the compound THC, an active ingredient of the psychoactive drug cannabis; and by synthetic analogues of THC.

The term “Cannabinoid receptor type 2 (CB2)” refers hereinafter to a G protein-coupled receptor from the cannabinoid receptor family that in humans is encoded by the CNR2 gene. It is closely related to the cannabinoid receptor type 1, which is largely responsible for the efficacy of endocannabinoid-mediated presynaptic-inhibition, the psychoactive properties of tetrahydrocannabinol, the active agent in marijuana, and other phytocannabinoids (natural cannabinoids). The principal endogenous ligand for the CB2 receptor is 2-arachidonoylglycerol (2-AG).

The term “nonpsychoactive” refers hereinafter not affecting the mind or mental processes.

The term “cannabinoid” refers hereinafter to a class of diverse chemical compounds that act on cannabinoid receptors on cells that repress neurotransmitter release in the brain. These receptor proteins include the endocannabinoids (produced naturally in the body by humans and animals), the phytocannabinoids (found in cannabis and some other plants), and synthetic cannabinoids.

The term “sustained release dosage form” refers hereinafter to the release of a drug at a predetermined rate in order to maintain a constant drug concentration for a specific period of time with minimum side effects. This can be achieved through a variety of formulations, including liposomes and drug-polymer conjugates. Sustained release in the present invention is akin to a “controlled release”.

The term “therapeutically effective amount” refers hereinafter to the amount of an agent or agents present at a sufficient concentration to produce a therapeutical effect on a patient, cells, or any combination thereof.

The term “multiple myeloma (MM) therapeutic agent” refers hereinafter to any agent from the family of alkylating agents, corticosteroids, proteasome inhibitors, immunomodulatory drugs, and any combination thereof, capable of negatively modulating, in a therapeutic manner, the development of MM.

The term “XTT cell proliferation kit” refers hereinafter to a colorimetric assay for analyzing the number of viable cells. The assay is based on the cleavage of the tetrazolium salt XTT in the presence of an electron-coupling reagent, producing a soluble formazan salt. This conversion only occurs in viable cells. Cells grown in a 96-well tissue culture plate are incubated with the XTT labeling mixture for 2-20 hours. After this incubation period, the formazan dye formed is quantitated using a scanning multi-well spectrophotometer (ELISA reader). The measured absorbance directly correlates to the number of viable cells.

The term “cytotoxic cocktail” refers hereinafter to a combination of compounds which have an inhibitory or cytotoxic effect on MM cells useful in treating MM.

The term “similar concentration” refers hereinafter to a concentration which is ±25% of the defined concentration value, preferably ±10% of the defined concentration value, more preferably ±5% of the defined concentration value.

The present invention provides a cytotoxic cocktail comprising: a therapeutically effective amount of at least one cannabinoid selected from the group consisting of: cannabidiol (CBD) or a derivative thereof, Tetrahydrocannabinol (THC) or a derivative thereof, and any combination thereof; and at least one therapeutic agent selected from the group consisting of: bortezomib (BTZ), carfilzomib (CFZ), lenalidomide (LEN), dexamethasone (DEX), melphalan (MEL) and doxorubicin (DOXO); wherein said cocktail is conferring a synergistic effect with respect to inhibition or cytotoxicity of multiple myeloma (MM) cells, relative to said at least one therapeutic agent selected from the group consisting of: BTZ, CFZ, LEN, DEX, MEL, DOXO and said CBD and THC, administered separately in a similar concentration.

The present invention further provides a cytotoxic cocktail characterized by: an effective amount of at least one cannabinoid selected from the group consisting of: cannabidiol (CBD) or a derivative thereof, Tetrahydrocannabinol (THC) or a derivative thereof and any combination thereof; at least one therapeutic agent selected from the group consisting of bortezomib (BTZ), carfilzomib (CFZ), lenalidomide (LEN), dexamethasone (DEX), melphalan (MEL) and doxorubicin (DOXO); said CBD and said THC are in a predefined ratio conferring a synergistic effect with respect to inhibition of multiple myeloma (MM) cells relative to said CBD and said THC administered separately in a similar concentration; wherein said cocktail conferring a synergistic effect with respect to inhibition or cytotoxicity of multiple myeloma (MM) cells relative to said at least one of BTZ, CFZ, LEN, DEX, MEL, DOXO, administered separately in a similar concentration.

The present invention further provides a method of treating multiple myeloma (MM) in a subject; said method comprising administrating to the subject a therapeutically effective amount of a cytotoxic cocktail consisting of: a therapeutically effective amount of at least one cannabinoid selected from a group consisting of: cannabidiol (CBD) or a derivative thereof, Tetrahydrocannabinol (THC) or a derivative thereof, and any combination thereof, and at least one of bortezomib (BTZ), carfilzomib (CFZ), lenalidomide (LEN), dexamethasone (DEX), melphalan (MEL), doxorubicin (DOXO); wherein said cocktail conferring a synergistic effect with respect to inhibition or cytotoxicity of multiple myeloma (MM) cells relative to said at least one of BTZ, CFZ, LEN, DEX, MEL, DOXO, CBD and THC administered separately in a similar concentration.

The present invention further provides a method of treating multiple myeloma (MM) in a subject; said method comprising administrating to said subject a therapeutically effective amount of cytotoxic cocktail characterized by: at least one cannabinoid selected from the group consisting of: cannabidiol (CBD) or a derivative thereof, Tetrahydrocannabinol (THC) or a derivative thereof, and any combination thereof; at least one of bortezomib (BTZ), carfilzomib (CFZ), lenalidomide (LEN), dexamethasone (DEX), melphalan (MEL), doxorubicin (DOXO); said THC and said CBD are administered in a predefined ratio providing a synergetic effect with respect to inhibition of multiple myeloma (MM) cells relative to said CBD and said THC when administered separately in a similar concentration; wherein said cocktail conferring a synergistic effect with respect to inhibition or cytotoxicity of multiple myeloma (MM) cells relative to said at least one of BTZ, CFZ, LEN, DEX, MEL, DOXO, administered separately in a similar concentration.

The present invention further provides a method for the treatment of MM cells by administering a therapeutic dose of the aforementioned cytotoxic cocktail to human MM cells. The cells can be in vitro, ex vivo, in situ, or others.

The present invention further provides the use of a cytotoxic cocktail comprising: a therapeutically effective amount of, or an extract consisting of essentially a therapeutically effective amount of at least one cannabinoid selected from the group consisting of: cannabidiol (CBD) or a derivative thereof, Tetrahydrocannabinol (THC) or a derivative thereof, and any combination thereof; at least one of bortezomib (BTZ), carfilzomib (CFZ), lenalidomide (LEN), dexamethasone (DEX), melphalan (MEL), doxorubicin (DOXO); in the manufacture of a medicament to treat multiple myeloma (MM); wherein said cocktail is conferring a synergistic effect with respect to inhibition or cytotoxicity of multiple myeloma (MM) cells, relative to said at least one therapeutic agent selected from the group consisting of: BTZ, CFZ, LEN, DEX, MEL, DOXO and said CBD and THC, administered separately in a similar concentration.

The present invention further provides a pharmaceutical composition comprising therapeutically effective amount of, or an extract consisting essentially therapeutically effective amount of at least one cannabinoid selected from the group consisting of: cannabidiol (CBD) or a derivative thereof, Tetrahydrocannabinol (THC) or a derivative thereof, and any combination thereof, for use in the treatment of multiple myeloma (MM).

According to one aspect of the present invention, the cannabidiol (CBD) or a derivative thereof and Tetrahydrocannabinol (THC) or a derivative thereof, of the composition of the present invention are acting as modulators of the endocannabinoid system activity. According to other aspects of the present invention, cannabinoids may cause alteration of the immune function, and induction of apoptosis in abnormal cells, while not affecting normal cells.

Without wishing to be bound by theory, the THC component of the composition of the present invention may function by enhancing the apoptotic impact of the CBD, while exerting antineoplastic and proapoptotic effects. It is further noted that a synergistic effect is provided by the use of both cannabinoids, namely THC and CBD, which is not achievable with either compound alone. According to a specific embodiment, a composition comprising predetermined ratio between the two cannabinoids is provided by the present invention to treat MM.

It is according to one embodiment, to provide a pharmaceutical composition comprising therapeutically effective amount of, or an extract consisting essentially therapeutically effective amount of at least one cannabinoid selected from the group consisting of: cannabidiol (CBD) or a derivative thereof, Tetrahydrocannabinol (THC) or a derivative thereof, and any combination thereof, for use in the treatment of multiple myeloma (MM).

The present invention further provides a pharmaceutical composition characterized by an effective amount of at least one cannabinoid selected from the group consisting of: cannabidiol (CBD) or a derivative thereof, Tetrahydrocannabinol (THC) or a derivative thereof and any combination thereof; said CBD and said THC are in a predefined ratio conferring a synergistic effect with respect to inhibition of multiple myeloma (MM) cells relative to CBD and THC administered separately in a similar concentration.

It is further within the scope to provide the pharmaceutical composition as defined in any of the above, wherein CBD and THC are in a predefined ratio of about 5:1 or 1:5 or 1;1, respectively.

It is further within the scope to provide the pharmaceutical composition as defined in any of the above, wherein the concentration of the CBD is in the range of about 2% to about 20%.

It is further within the scope to provide the pharmaceutical composition as defined in any of the above, wherein the concentration of the THC or the derivative thereof is in the range of about 2% to about 20%.

It is further within the scope to provide the pharmaceutical composition as defined in any of the above, wherein the composition is adapted to be administered in a route selected from a group consisting of: intranasal, transdermal, intravenous, oral, and any combination thereof.

It is further within the scope to provide the pharmaceutical composition as defined in any of the above, wherein the composition is adapted for oral administration in a formulation selected from a group of preparations consisting of syrup, drops, tincture, tablet, capsule, solution, emulsion, suspension, granules, powder, and any combination thereof.

It is further within the scope to provide the pharmaceutical composition as defined in any of the above, wherein the composition is adapted to be administered in combination with an additional MM therapeutic agent.

It is further within the scope to provide the pharmaceutical composition as defined in any of the above, wherein the additional MM therapeutic agent is selected from a group consisting of alkylating agents, corticosteroids, proteasome inhibitors, and immunomodulatory drugs, and any combination thereof.

It is further within the scope to provide the pharmaceutical composition as defined in any of the above, wherein the additional MM therapeutic agent is selected from a group consisting of bortezomib (BTZ), lenalidomide (LEN), dexamethasone (DEX), melphalan (MEL), doxorubicin, and any combination thereof.

It is further within the scope to provide the pharmaceutical composition as defined in any of the above, wherein the CBD or the derivative thereof interacts with at least one receptor selected from a group consisting of Cannabinoid receptor type 1 (CB1), Cannabinoid receptor type 2 (CB2), and any combination thereof.

It is further within the scope to provide the pharmaceutical composition as defined in any of the above, wherein the THC or the derivative thereof interacts with at least one receptor selected from a group consisting of Cannabinoid receptor type 1 (CB1), Cannabinoid receptor type 2 (CB2), and any combination thereof.

It is further within the scope to provide the pharmaceutical composition as defined in any of the above, wherein the composition additionally comprises inactive ingredients selected from a group consisting of antiadherants, binders, coatings, disintegrants, flavours, colourants, lubricants, glidants, sorbents, preservatives, sweeteners, and any combination thereof.

It is further within the scope to provide the pharmaceutical composition as defined in any of the above, wherein the composition is in a sustained release dosage form; the sustained release dosage form is selected from a group consisting of liposomes, drug polymer conjugates, microencapsulation, controlled-release tablet coating, and any combination thereof.

It is further within the scope to provide the pharmaceutical composition as defined in any of the above, wherein the composition is nonpsychoactive.

It is further within the scope to provide the pharmaceutical composition as defined in any of the above, wherein the composition is administered once, twice, three or four times through the day.

It is further within the scope to provide the pharmaceutical composition as defined in any of the above, wherein the composition is obtained from at least one cannabis plant.

It is further within the scope to provide the pharmaceutical composition as defined in any of the above, wherein the cannabis plant is a CBD rich strain.

It is further within the scope to provide the pharmaceutical composition as defined in any of the above, wherein the CBD rich strain is selected from a group consisting of Avidekel, Fedora 17, ACDC, and any combination thereof.

It is further within the scope to provide the pharmaceutical composition as defined in any of the above, wherein the cannabis plant is a THC rich strain.

It is further within the scope to provide the pharmaceutical composition as defined in any of the above, wherein the THC rich strain is selected from a group consisting of Black Destroyer, Critical Neville Haze, Mataro Blue, LSD OG Kush, Pineapple Chunk, Blue Monster Holk, Y Griega, Satori, Tutankhamon, and any combination thereof.

It is further within the scope to provide the pharmaceutical composition as defined in any of the above, wherein the CBD or derivative thereof is produced by a synthetic route.

It is further within the scope to provide the pharmaceutical composition as defined in any of the above, wherein the THC or derivative thereof is produced by a synthetic route.

It is further within the scope to provide the pharmaceutical composition as defined in any of the above, wherein the composition is dissolved in a lipophilic solvent or suspension carrier.

It is further within the scope to provide the pharmaceutical composition as defined in any of the above, wherein the lipophilic solvent or suspension carrier are selected from a group consisting of medium-chain triglyceride, short-chain triglyceride, medium-chain partial glyceride, polyoxyethylated fatty alcohol, polyoxyethylated fatty acid, polyoxyethylated fatty acid triglyceride or partial glyceride, ester of fatty acids with low molecular weight alcohols, a partial ester of sorbitan with fatty acids, a polyoxyethylated partial ester of sorbitan with fatty acids, a partial ester of sugars or oligomeric sugars with fatty acids, a polyethylene glycol, lecithin, vegetable oil, and any combination thereof.

It is further within the scope to provide the pharmaceutical composition as defined in any of the above, wherein CBD and THC administered in a ratio of about 1:1, respectively confers a synergistic effect with respect to inhibition of multiple myeloma (MM) cells relative to the CBD and the THC administered separately in a similar concentration.

It is further within the scope to provide the pharmaceutical composition as defined in any of the above, wherein CBD and THC administered in a ratio of about 1:5, respectively confers a synergistic effect with respect to inhibition of multiple myeloma (MM) cells relative to the CBD and the THC administered separately in a similar concentration.

It is further within the scope to provide the pharmaceutical composition as defined in any of the above, wherein CBD and THC administered in a ratio of about 5:1, respectively confers a synergistic effect with respect to inhibition of multiple myeloma (MM) cells relative to CBD and THC administered separately in a similar concentration.

It is further within the scope to provide the pharmaceutical composition as defined in any of the above, wherein the synergistic effect is defined as at least 50% inhibition on RPMI8226 multiple myeloma (MM) cells in vitro.

It is further within the scope to provide the pharmaceutical composition as defined in any of the above, wherein the synergistic effect is defined as more than about 80% inhibition on RPMI8226 multiple myeloma (MM) cells in vitro.

It is further within the scope to provide the pharmaceutical composition as defined in any of the above, wherein CBD and THC have a combination index (CI) value of less than 1 indicating synergism.

It is according to another embodiment, to provide a method of treating multiple myeloma (MM) in a subject; the method comprising administrating to the subject a therapeutically effective amount of, or an extract consisting essentially therapeutically effective amount of at least one cannabinoid selected from the group consisting of: cannabidiol (CBD) or a derivative thereof, Tetrahydrocannabinol (THC) or a derivative thereof, and any combination thereof.

It is according to another embodiment, to disclose the use of a composition comprising a therapeutically effective amount of, or an extract consisting essentially a therapeutically effective amount of at least one cannabinoid selected from the group consisting of: cannabidiol (CBD) or a derivative thereof, Tetrahydrocannabinol (THC) or a derivative thereof, and any combination thereof in the manufacture of a medicament to treat multiple myeloma (MM).

It is further within the scope to provide the use of a composition as defined in any of the above, wherein CBD and THC administered in a ratio of about 1:1, respectively confers a synergistic effect with respect to inhibition of multiple myeloma (MM) cells relative to CBD and THC administered separately in a similar concentration.

It is further within the scope to provide the use of a composition as defined in any of the above, wherein CBD and THC administered in a ratio of about 1:5, respectively confers a synergistic effect with respect to inhibition of multiple myeloma (MM) cells relative to CBD and THC administered separately in a similar concentration.

It is further within the scope to provide the use of a composition as defined in any of the above, wherein CBD and THC administered in a ratio of about 5:1, respectively confers a synergistic effect with respect to inhibition of multiple myeloma (MM) cells relative to CBD and THC administered separately in a similar concentration.

It is further within the scope to provide the use of a composition as defined in any of the above, wherein the synergistic effect is defined as at least 50% inhibition on RPMIS multiple myeloma (MM) cells in vitro.

It is further within the scope to provide the use of a composition as defined in any of the above, wherein the synergistic effect is defined as more than about 80% inhibition on RPMIS multiple myeloma (MM) cells in vitro.

It is further within the scope to provide the use of a composition as defined in any of the above, wherein CBD and THC have a combination index (CI) value of less than 1 indicating synergism.

In order to understand the invention and to see how it may be implemented in practice, a plurality of preferred embodiments will now be described, by way of non-limiting example only, with reference to the following examples.

### EXAMPLE 1

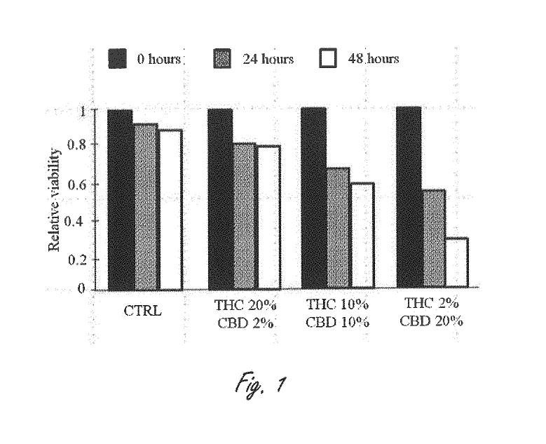

Reference is now made to FIG. 1 which demonstrates a graph of the relative viability of MM cells vs. different concentrations of CBD and THC, during different time periods (i.e 0, 24 and 48 hours). The effect of different concentrations of CBD and THC on the viability of different multiple myeloma cell lines and primary cells isolated from bone marrow of myeloma patients in the presence and absence of bone marrow stroma cells, was tested. Several MM cell lines were plated at 2×10⁴ cells per well in 96-wells and reacted with different concentrations of CBD and THC. Samples were taken from bone marrow aspirates from MM patients. Mononuclear cells were separated by Ficoll density gradient centrifugation and myeloma cells selected using CD138 microbeads (Miltenyi Biotec). Purified CD138+ patient cells were be plated at a density of 2×10⁴ cells per well and treated for 48 hours with different concentrations of CBD and THC (THC 2% CBD 20%; THC 10% CBD 10%; and THC 20% CBD 2%). Cell viability was measured using XTT cell proliferation Kit (Biological Industries) according to manufacture instructions. It can be seen from FIG. 1, that in comparison to the control sample (in which only buffer was added) all combinations of CBD and THC showed an effect upon the viability of the cells.

### EXAMPLE 2

Combinations of novel and/or conventional anti-MM agents can achieve higher clinical response rates than single agent(s). In addition, many patients experience significant dose-limiting side effects requiring dose reductions or cessation of therapy.

The anti-MM activity induced by the combination of CBD and THC (THC 2% CBD 20%; THC 10% CBD 10%; and THC 20% CBD 2% with other MM chemotherapeutic drugs in vitro was assessed. The response of MM cells to treatment with CBD and THC in combination with currently in use anti-MM agents (bortezomib (BTZ), lenalidomide (LEN), dexamethasone (DEX), melphalan (MEL) and doxorubicin (DOXO) were evaluated. The anti-MM activity of combined treatment was analyzed by XTT assays as previously described in example 1, and the presence of synergistic cytotoxic effects are be evaluated using the Chou-Talalay method based on the median-effect equation and the classic isobologram equation and compusyn computer software. It appears that in comparison to the control (in which only buffer was added) or to currently in use anti-MM agents, all combinations of CBD and THC affected the viability of the cells.

### EXAMPLE 3

This example presents the mode of action of cannabis as an anti-myeloma agent, the effect of cannabis on MM cell lines is evaluated regarding apoptosis, angiogenesis, cell cycle, mitochondrial transmembrane potential, ROS production, and cell signaling.

Apoptosis analysis: MM cells were treated with different concentrations of CBD and THC (THC 2% CBD 20%; THC 10% CBD 10%; and THC 20% CBD 2%) during 0, 24 and 48 h. For evaluation of apoptosis, cells were processed using an Annexin V/propidium iodide (PI) kit (Becton Dickinson Biosciences) according to manufacture instructions.

Cell-cycle analysis: MM cells were exposed to different concentrations of CBD and THC (THC 2% CBD 20%; THC 10% CBD 10%; and THC 20% CBD 2%) for different intervals of time. The cells were then permeabilized by 70% ethanol at −20° C. overnight and incubated with 50 μg/ml PI and 20 units/ml RNase-A (Roche Diagnostics). DNA content was analyzed by flow cytometry. Data was collected using FACS Calibur (Becton Dickinson) and analyzed with the CellQuest software.

Cell signaling: MM cell lines were plated in RPMI 1640 with 10% FBS, penicillin, and streptomycin. CBD and THC (THC 2% CBD 20%; THC 10% CBD 10% and; THC 20% CBD 2%) was added after 0, 30 minutes, 2, 6, 24 and 48 h. Cells were then lysed in RIPA-lysis buffer (10 mM sodium pyrophosphate, 2 mM sodium orthovanadate, 5 mM sodium fluoride, 5 g/mL aprotinin, 5 g/mL leupeptin, and 1 mM phenylmethylsulfonyl fluoride). Proteins were separated by sodium dodecyl sulfate-polyacrylamide gel electrophoresis, transferred onto nitrocellulose membranes and immunoblotted with cell signaling antibodies. Immunoreactive bands were detected by Western Blot chemiluminescence reagents (Thermo Scientific) and exposed on Kodak-XAR film.

Cell signaling arrest was achieved with the THC and CBD extract combinations, with different THC and CBD ratios providing different levels of arrest.

Mitochondrial transmembrane potential: Mitochondrial transmembrane potential (Dwm) was evaluated by 5, 5′,6,6′-tetrachloro-1,1′,3,3′-tetraehylbenzimidazolylcarbocyanineiodide (JC-1) staining. Briefly, 2×104 cells were treated with of CBD and THC (THC 2% CBD 20%; THC 10% CBD 10%; and THC 20% CBD 2%) for different times and then incubated for 10 min at room temperature with 10 μg/ml of JC-1. JC-1 was excited by an argon laser (488 nm), and the green (530 nm)/red (570 nm) emission fluorescence was collected simultaneously. Carbonyl cyanide chlorophenylhydrazone protonophore, a mitochondrial uncoupler that collapses (Dwm), was be used as a positive control. Samples were analyzed using a FACScan cytofluorimeter with CellQuest software. Different levels of reduction arrest in mitochondrial transmembrane potential were achieved with the various THC and CBD extracts of the present invention.

ROS production: The fluorescent probe dichlorodihydrofluorescein diacetate (DCFDA) was used to assess oxidative stress levels. Briefly, 2×104 cells treated with the appropriate compounds were incubated with 20 μM DCFDA (Life Technologies Italia, Italy) 20 min prior to the harvest time point. The cells were then washed, and the intensity of the fluorescence was assayed using flow cytometry and CellQuest software. Different levels of reduction arrest ROS production was obtained with the THC and CBD extracts herein described.

### EXAMPLE 4

This example presents the effect of cannabis on osteoblasts (OB) function, MC3T3-E1 pre-osteoblastic cells (ATCC) and bone marrow-derived stromal cells were cultured in osteoblastic differentiation media, with or without MM cells, in the presence of different concentrations of CBD and THC; (THC 2% CBD 20%; THC 10% CBD 10%; and THC 20% CBD 2%) for different periods of time. At the end of the culture period, cells will be evaluated for OB differentiation. To evaluate the effect of cannabis on OC function, mononuclear cells from MM patients were differentiated to osteoclasts and treated with cannabis and their activity evaluated in the presence and absence of stroma cells.

### EXAMPLE 5

This example examines anti-tumor efficacy of cannabis in murine xenograft MM model SCID mice (6-8 week old) were maintained in accordance with Institutional Animal Care Use Committee guidelines. Mice were gamma-irradiated (150 rads) using Cs137 γ-irradiator source and (24 hours post-irradiation) injected subcutaneously with MM cells (7×106/mouse) suspended in PBS. 2-3 weeks later, when palpable tumors were developed, mice were be randomly divided into different groups (15 mice/group), and the following treatment protocol will be implemented: Group 1a-b*: vehicle control administered ip, every day, 5 days a week throughout the duration of the experiment; Group 2-4 a-b*: the best combination of THC 2% CBD 20%, CBD 10% THC 10% or THC 20% CBD 2% according to in vitro results at different doses (1, 10 and 20 mg/kg) administered ip, every day, 5 days a week throughout the duration of experiment; Group 5-6 a-b*: THC and CBD at 20 mg/kg administered ip, every day, 5 days a week throughout the duration of experiment; * Subgroup a: mice treated during 3 weeks (n=5) and tumor removed and analyzed *Subgroup b: mice treated until the end of the experiment (n=15). Evaluation of efficacy will include inhibition of tumor growth, survival, blood tests, animals' vital signs and gross pathology. Tumor size will be measured by caliper. Caliper measurements of the longest perpendicular tumor diameters will be performed every other day to estimate tumor volume. All compositions showed decrease in tumor size but the highest reduction was shown in the solution having THC 2% CBD 20%.

### EXAMPLE 6

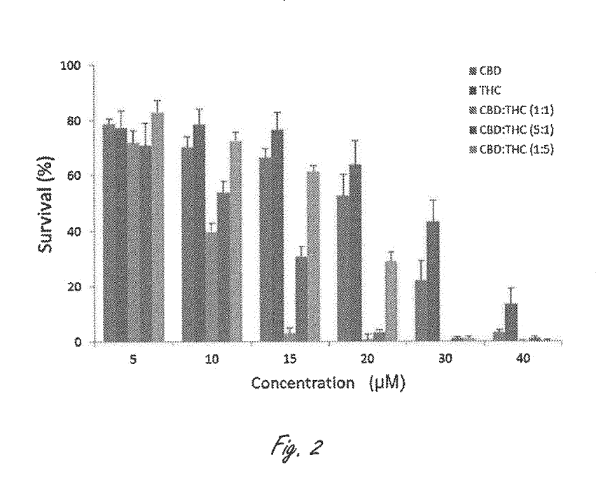

This example examines the cytotoxic effect of CBD alone, THC alone and combinations of both compounds. The cytotoxic effect of CBD, THC and their combinations in different ratios such as CBD: THC 1:1; CBD: THC 5:1 and CBD: THC 1:5 were evaluated on RPMI8226 multiple myeloma (MM) human cell lines. Reference is now made to FIG. 2 which presents a graph of RPMIS MM cell line survival (%) vs. concentration (μM).

As illustrated in FIG. 2, CBD and THC, and their combinations decreased the survival of MM cells in a concentration dependent manner. The dose that caused 50% of MM cell death was 16 μM and 22 μM for CBD and THC, respectively.

It is demonstrated in this figure that treatment with CBD in combination with THC had synergistic effects, the cytotoxic effect being higher with each of the three combinations tested, relative to treatment with CBD or THC separately.

Furthermore, in a concentration of about 15 μM and more, the cytotoxic effect of CBD and THC combinations (e.g. CBD: THC 1:1; CBD: THC 5:1) has demonstrated less than 30% survival of RPMIS MM cells, while treatment with CBD or THC separately demonstrated higher than about 70% survival rate of the RPMIS MM cells. Moreover, in a concentration of about 20 μM and higher, the cytotoxic effect of all CBD and THC combinations (i.e. CBD: THC 1:1; CBD: THC 5:1 and CBD: THC 5:1), demonstrated less than 30% survival of RPMIS MM cells, while treatment with CBD or THC separately gave about 50% survival rate of the RPMIS MM cells. Thus, this experiment demonstrates the significantly higher cytotoxic effect of CBD and THC combinations as compared to their effect when administered separately.

### EXAMPLE 7

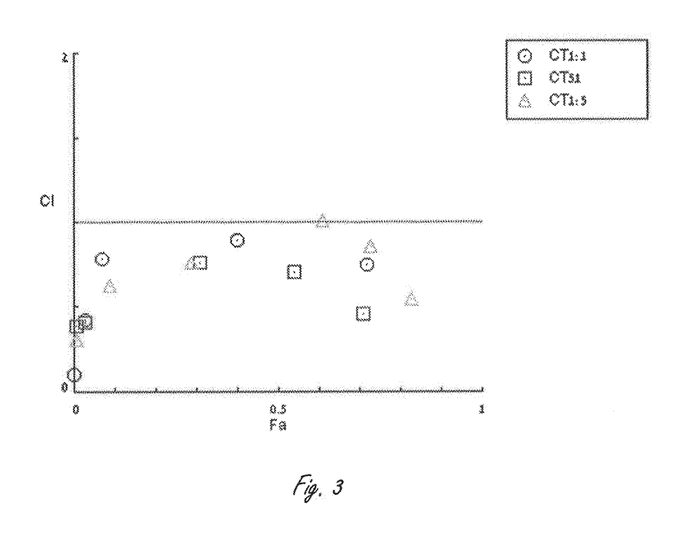

This experiment shows the combinatorial effect of CBD when administered together with THC. Reference is now made to FIG. 3 which presents a graph of the ratio of the THC and/or CBD fraction affected (Fa) vs. the Combination Index (CI). The graph demonstrates the effect of the combination of CBD with THC upon RPMI8226 MM cells. RPMIS cells were cultured for 48 hours with CBD and THC and compared to their combinations (i.e. CBD: THC 1:1; CBD: THC 5:1 and CBD: THC 1:5).

As illustrated in FIG. 3, the CI value <1, CI=1 and CI>1 indicates quantitative definition of synergism, additive effect, and antagonism, respectively. Each treatment was performed in triplicate in four independent experiments and presented as mean±SE.

It is shown that the combination of CBD and THC in the ratio of 1:1 is with CI less than 0.9. In another exemplary embodiment, the combination of CBD and THC in the ratio of 5:1 is with CI less than 0.7. The different ratios of the combination of CBD and THC (i.e. CBD: THC 1:1; CBD: THC 5:1 and CBD: THC 1:5) demonstrate CI<1 thereby, exhibiting synergy.

### EXAMPLE 8

Cytotoxic effect of CBD, THC, CBD: THC (1:1; 5:1, 1:5 and 5:2 respectively) in combination with conventional and novel anti-MM therapies on RPMI8266 multiple myeloma (MM) cells.

Today, most MM patients are treated with two or -three drug combination therapy, to achieve the an effective response. Therefore, the viability of RPMI8226 MM cells after being treated with CBD, THC, CBD: THC (1:1; 5:1, 1:5 and 5:2 respectively) in combination with the currently in use anti-MM drugs including: the proteasome inhibitors bortezomib (BTZ), carfilzomib (CFZ), corticosteroids (DEX), alkylating agents (MEL) and intercalating DNA agents (DOXO) was assessed.

The cells were treated during 48 hours with different concentrations of each drug alone or in combination with CBD, THC, CBD: THC (1:1; 5:1, 1:5 and 5:2 respectively) and their viability evaluated by XTT assay.

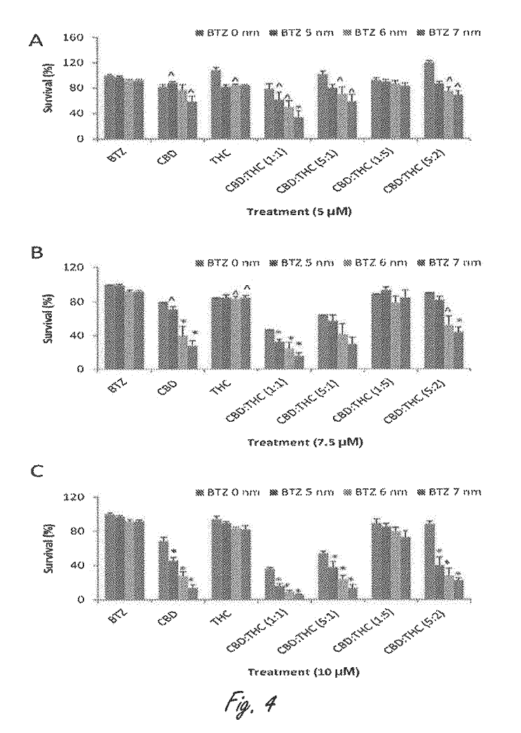

Reference is now made to FIG. 4 which presents the cytotoxic effects of CBD, THC and CBD: THC (1:1; 5:1, 1:5 and 5:2 respectively) in combination with BTZ. As can be seen the combination of CBD and CBD: THC (1:1; 5:1, 5:2 respectively) with BTZ decreased significantly (p<0.0001) the viability of RPMI8226 cells as compared to BTZ alone. Importantly, the effect of CBD: THC (1:1) was significantly higher than that of CBD alone (p<0.01). The addition of BTZ had no observable effect with THC and CBD: THC (1:5) treatment. The cytotoxic effect of CBD: THC (1:1) in combination with BTZ was dose dependent, (n=3 in three independent experiments).

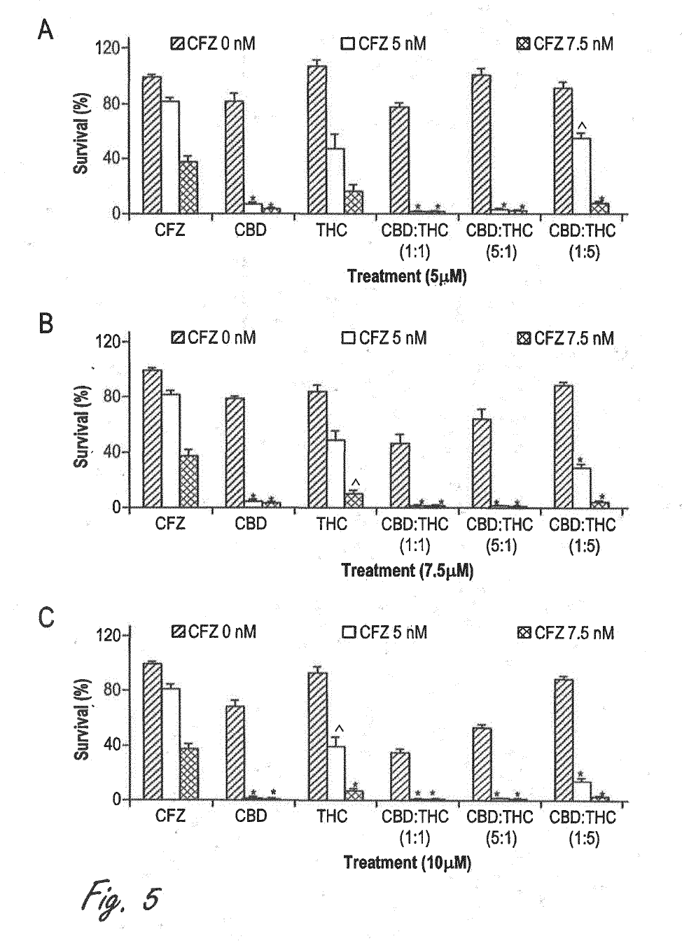

Reference is now made to FIG. 5 which presents the cytotoxic effects of CBD, THC and CBD: THC (1:1; 5:1, 1:5 respectively) in combination with CFZ. As can be seen the combination of CBD and CBD: THC (1:1; 5:1, 5:2 respectively) with CFZ decreased significantly (p<0.0001) the viability of RPMI8226 cells as compared to CFZ alone. Importantly, the effect of CBD: THC (1:1) was significantly higher than that of CBD alone (p<0.001). The addition of CFZ had no observable effect with THC and CBD: THC (1:5) treatment, (n=3 in three independent experiments).

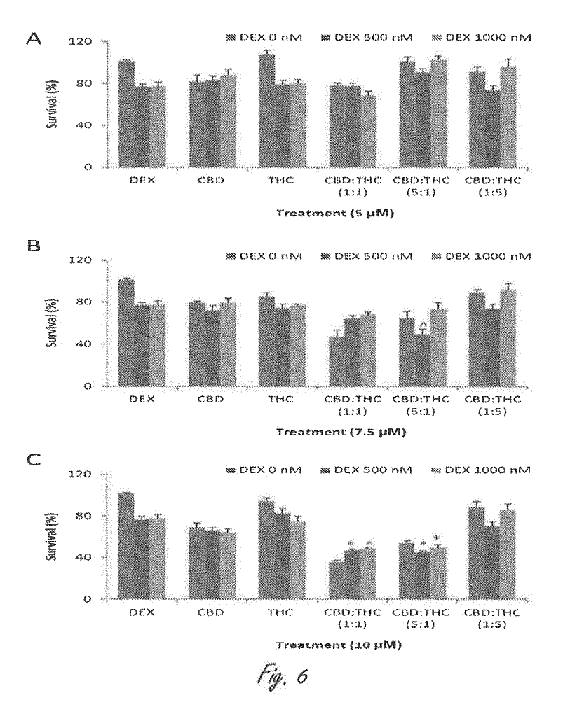

Reference is now made to FIG. 6 which presents the cytotoxic effects of CBD, THC and CBD: THC (1:1; 5:1, 1:5 respectively) in combination with DEX. As can be seen the combination CBD: THC (1:1; 5:1 respectively) with DEX decreased significantly (p<0.0001) the viability of RPMI8226 cells as compared to DEX alone in a dose dependent manner. The combination of CBD, THC and CBD: THC (1:5) with DEX had not increased their toxicity effect on RPMI8226 cells, (n=3 in three independent experiments).

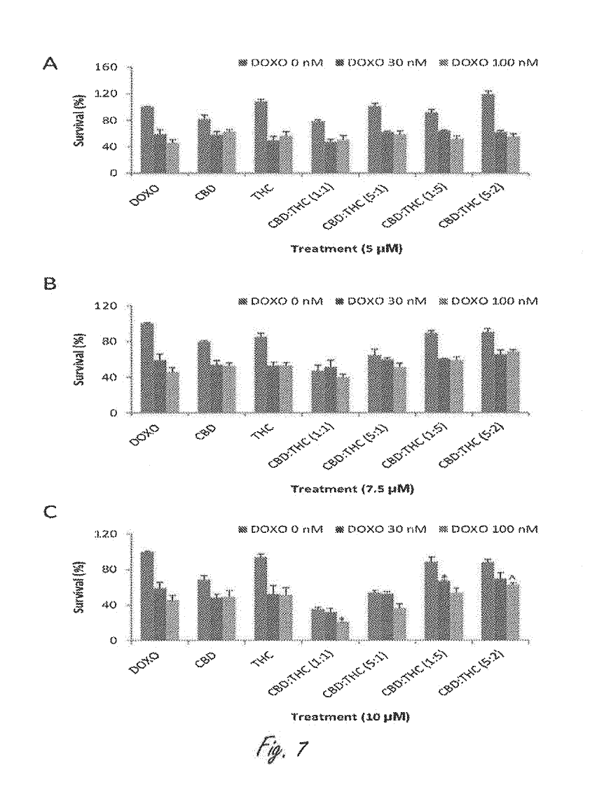

Reference is now made to FIG. 7 which presents the cytotoxic effects of CBD, THC and CBD: THC (1:1; 5:1, 1:5 and 5:2 respectively) in combination with DOXO. As can be seen the combination CBD: THC (1:1 respectively) with DOXO decreased significantly (p<0.0001) the viability of RPMI8226 cells as compared to DOXO or CBD: THC (1:1) alone in a dose dependent manner. The combination of CBD, THC and CBD: THC (5:1, 5:2 and 1:5 respectively) with DOXO had no increased toxicity on RPMI8226 cells (FIG. 3), (n=3 in one experiment).

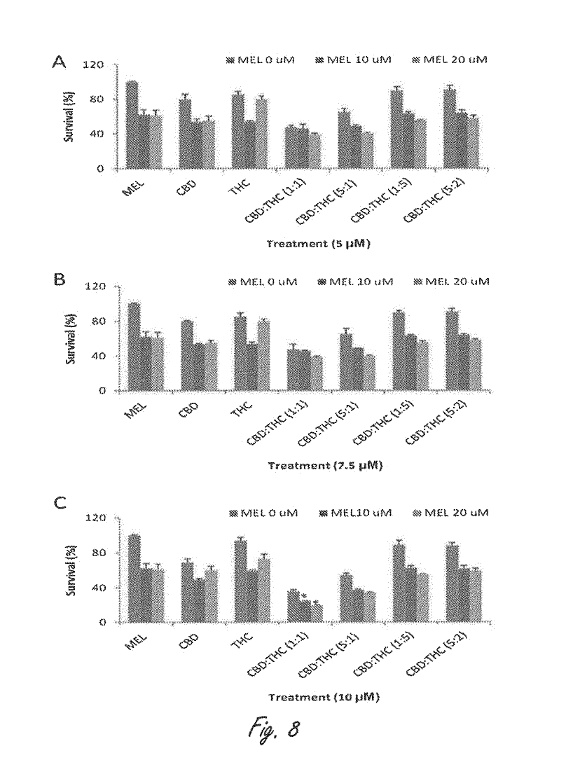

Reference is now made to FIG. 8 which presents the cytotoxic effects of CBD, THC and CBD: THC (1:1; 5:1, 1:5 and 5:2 respectively) in combination with MEL. As can be seen the combination CBD: THC (1:1 respectively) with MEL decreased significantly (p<0.0001) the viability of RPMI8226 cells as compared to DOXO, MEL or CBD: THC (1:1) alone in a dose dependent manner. The combination of CBD, THC and CBD: THC (5:1, 5:2 and 1:5 respectively) with MEL had no increased toxicity on RPMI8226 cells (FIG. 3), (n=3 in one experiment).

In conclusion: the combination of CBD: THC (1:1) with BTZ, CFZ, DOXO and MEL results in a significantly increased cytotoxicity as compared to BTZ, CFZ, DOXO, MEL, CBD and THC alone. CBD and CBD: THC (1:1; 5:1, 5:2 respectively) in combination with BTZ and CFZ also decreased significantly the viability of RPMI8226 cells in comparison with BTZ, CFZ, CBD and THC alone.

1. A cytotoxic cocktail comprising:
a. a therapeutically effective amount of at least one cannabinoid selected from the group consisting of: cannabidiol (CBD) or a derivative thereof, Tetrahydrocannabinol (THC) or a derivative thereof, and any combination thereof, and
b. at least one therapeutic agent selected from the group consisting of: bortezomib (BTZ), carfilzomib (CFZ), lenalidomide (LEN), dexamethasone (DEX), melphalan (MEL) and doxorubicin (DOXO);
wherein said cocktail is conferring a synergistic effect with respect to inhibition or cytotoxicity of multiple myeloma (MM) cells, relative to said at least one therapeutic agent selected from the group consisting of: BTZ, CFZ, LEN, DEX, DOXO and said CBD and THC, administered separately in a similar concentration.

2. The cytotoxic cocktail of claim 1, wherein said CBD and said THC are in a predefined ratio conferring inhibition or cytotoxicity of multiple myeloma (MM) cells relative to said CBD and said THC administered separately in a similar concentration.

3. The cytotoxic cocktail of claim 1, wherein said CBI) and said THC are in a predefined ratio conferring a synergistic effect with respect to inhibition or cytotoxicity of multiple myeloma (MM) cells relative to said CBD and said THC administered separately in a similar concentration.

4. The cytotoxic cocktail of claim 3, wherein at least one of the following holds true:
a. said ratio is selected from the group consisting of: about 1:1,about 1:5, about 5:1, about 5:2, respectively;
b. said synergistic effect is defined as art least 50% inhibition of RPMIS multiple myeloma (MM) cells in vitro;
c. said cytotoxic or inhibition effect is defined as significantly decreased viability of RPMIS multiple myeloma (MM) cells in vitro, said decrease in viability is defined as decreased viability of at least 50% of RPMIS multiple myeloma (MM) cells in vitro; or
d. said CBD and said THC have a combination index (CI) value lower than 1 indicating synergism.

5.-12. (canceled)

13. The cytotoxic cocktail of claim 1, wherein at least one of the following holds true:
a. said at least one of BTZ, CFZ, LEN, DEX, MEL and DOXO in said cocktail decreases the viability of MM cells, in a dose dependent manner;
b. the concentration of said CBD is in the range of about 2% to about 20%; or
c. the concentration of said THC or said derivative thereof is in the range of about 2% to about 20%.

14. (canceled)

15. The cytotoxic cocktail of claim 1, wherein said composition is adapted to be administered in a route selected from a group consisting of: intranasal, transdermal, intravenous, oral, and any combination thereof.

16. The cytotoxic cocktail of claim 15, wherein said composition is adapted for oral administration in a formulation selected from a group of preparations consisting of syrup, drops, tincture, tablet, capsule, solution, emulsion, suspension, granules, powder, and any combination thereof.

17. The cytotoxic cocktail of claim 1, wherein said composition is adapted to be administered in combination with an additional MM therapeutic agent.

18. The cytotoxic cocktail of claim 17, wherein said additional MM therapeutic agent is selected from a group consisting of alkylating agents, corticosteroids, proteasome inhibitors, immunomodulatory drugs, and any combination thereof.

19. The cytotoxic cocktail of claim 1, wherein at least one of the following holds true:
a. said CBD or said derivative thereof interacts with at least one receptor selected from the group consisting of Cannabinoid receptor type 1 (CB1), Cannabinoid receptor type 2 (CB2), and any combination thereof; or
b. said THC or said derivative thereof interacts with at least one receptor selected from the group consisting of Cannabinoid receptor type 1 (CB1), Cannabinoid receptor type 2 (CB2), and any combination thereof.

20. (canceled)

21. The cytotoxic cocktail of claim 1, wherein at least one of the following holds true:
a. said composition additionally comprises inactive ingredients selected from the group consisting of antiadherants, binders, coatings, disintegrants, flavours, colours, lubricants, glidants, sorbents, preservatives, sweeteners, and any combination thereof;
b. said composition is in a sustained release dosage form selected from the group consisting of liposomes, drug polymer conjugates, microencapsulation, controlled-release tablet coating, and any combination thereof;
c. said composition is nonpsychoactive;
d. said composition is administered once, twice, three or four times through the day; or
e. said CBD and/or said THC is obtained from at least one cannabis plant.

22.-25. (canceled)

26. The cytotoxic cocktail of claim 21, wherein at least one of the following holds true:
a. said cannabis plant is a CBD rich strain; or
b. said cannabis plant is a THC rich strain.

27-29. (canceled)

30. The cytotoxic cocktail of claim 1, wherein at least one of the following holds true:
a. said composition comprises a cannabis extract or cannabis oil;
b. said THC or derivative thereof and/or said CBD or derivative thereof is derived from a cannabis extract;
c. said CBD or derivative thereat is produced by a synthetic route;
d. said THC or derivative thereof is produced by a synthetic route; or
e. said composition is dissolved in a lipophilic solvent or suspension carrier.

31-34. (canceled)

35. The cytotoxic cocktail of claim 30, wherein said lipophilic solvent or suspension carrier are selected from a group consisting of medium-chain triglyceride, short-chain triglyceride, medium-chain partial glyceride, polyoxyethylated fatty alcohol, polyoxyethylated fatty acid, polyoxyethylated fatty acid triglyceride or partial glyceride, ester of fatty acids with low molecular weight alcohols, a partial ester of sorbitan with fatty acids, a polyoxyethylated partial ester of sorbitan with fatty acids, a partial ester of sugars or oligomeric sugars with fatty acids, a polyethylene glycol, lecithin, vegetable oil, and any combination thereof.

36. The cytotoxic cocktail of claim 1, wherein said cocktail decreases significantly the viability of RPMI8226 cells in comparison to BTZ, CFZ, CBD and THC alone.

37. The cytotoxic cocktail of claim 1, wherein the combination of CBD: THC (1:1) with BTZ, CFZ, DOXO and MEL results in a significantly increased cytotoxicity effect as compared to BTZ, CFZ, DOXO, MEL, CBD and THC alone.

38. The cytotoxic cocktail of claim 1, wherein the combination of CBD or CBD: THC (1:1; 5:1, 5:2 respectively) with BTZ or CFZ decreases significantly the viability of RPMI8226 cells in comparison with BTZ, CFZ, CBD and THC alone.

39.-43. (canceled)

44. A method of treating multiple myeloma (MM) in a subject; said method comprising administrating to the subject a therapeutically effective amount of a cytotoxic cocktail comprising:
a. a therapeutically effective amount of at least one cannabinoid selected from a group consisting of: cannabidiol (CBD) or a derivative thereof, Tetrahydrocannabinol (THC) or a derivative thereof, and any combination thereof, and
b. at least one therapeutic agent consisting of bortezomib (BTZ), carfilzomib (CFZ), lenalidomide (LEN), dexamethasone (DEX), melphalan (MEL) and doxorubicin (DOXO); wherein said cocktail is conferring a synergistic effect with respect to inhibition or cytotoxicity of multiple myeloma (MM) cells relative to said at least one of BTZ, CFZ, LEN, DEX, MEL, DOXO, CBD and THC administered separately in a similar concentration.

45. The method of claim 44, additionally comprising at least one step of:
a. providing said CBD and said TUC in a predefined ratio of about 1:5 or 5:1, 5:2 or 1:1, respectively; or
b. administrating said at least one of BTZ, CFZ, LEN, DEX, MEL and DOXO significantly decreases the viability of MM cells in a dose dependent manner.

46. The method of claim 45, wherein said step of administrating said CBD and said THC in said predefined ratio confers a synergistic effect with respect to inhibition of multiple myeloma (MM) cells relative to said CBD and said THC when administered separately in a similar concentration.

47. (canceled)

48. The method of claim 44, additionally comprising at least one step of:
a. providing said extract with CBD concentration in the range of about 2% to about 20%;
b. providing said extract with THC concentration in the range of about 2% to about 20%;
c. administering said composition in a route selected from the group consisting of: intranasal, transdermal, intravenous, oral, and any combination thereof;
d. administering said composition orally in a formulation selected from a group of preparations consisting of syrup, drops, tincture, tablet, capsule, solution, emulsion, suspension, granules, powder, and any combination thereof;
e. administering said composition in a manner selected from a group consisting of: intranasal, transdermal, intravenous, oral, and any combination thereof;
f. administering said composition over a time period of about 1 day to about 6 months;
g. administering said composition in a dosage of CBD of up to 400 mg per day, preferably in the range of about 2 mg to about 400 mg per day;
h. administering said composition in a dosage THC of up to 400 mg per day, preferably in the range of about 10 mg to about 400 mg per day;
i. administering said composition once, twice, three or four times through the day;
j. administering said composition with an additional MM therapeutic agent selected from the group consisting of alkylating agents, corticosteroids, proteasome inhibitors, immunomodulatory drugs, and any combination thereof;
k. formulating said composition with an inactive ingredient selected from a group consisting of antiadherents, binders, coatings, disintegrants, flavours, colours, lubricants, glidants, sorbents, preservatives, sweeteners, and any combination thereof;
l. formulating said composition in a sustained release dosage form selected from the group consisting of liposomes, drug polymer conjugates, microencapsulation, controlled-release tablet coating, and any combination thereof;
m. administering said CBD with Tetrahydrocannabinol (THC) in a concentration which is equal or less than 2%; or
n. administering said cocktail decreases significantly the viability of RPMI8226 cells in comparison with BTZ, CFZ, CBD and THC alone.

49.-70. (canceled)

71. A method for inhibiting multiple myeloma (MM) cells by administering a therapeutic dose of a cytotoxic cocktail according to claim 1 to human MM cells.

72. The method of claim 71, wherein said MM cells are selected from the group consisting of: in vitro, ex vivo and in situ cells.

73.-101. (canceled)
